(19) United States
(12) Patent Application Publication
(10) Pub. No.: US 2013/0252956 A1
(43) Pub. Date: Sep. 26, 2013

(54) METHODS OF TREATING CANCER

(71) Applicants: Howard Kallender, Collegeville, PA (US); Lone Ottesen, Uxbridge (GB)

(72) Inventors: Howard Kallender, Collegeville, PA (US); Lone Ottesen, Uxbridge (GB)

(21) Appl. No.: 13/988,561

(22) PCT Filed: Nov. 21, 2011
(86) PCT No.: PCT/US11/61636

§ 371 Date: May 21, 2013

Related U.S. Application Data
(60) Provisional application No. 61/415,994, filed on Nov. 22, 2010.

(51) Int. Cl. A61K 31/5377

(52) U.S. Cl. CPC A61K 31/5377
USPC 514/235.2

(57) ABSTRACT

Methods for treating a human having heptocellular carcinoma comprising administering to the human a therapeutically effective amount of Compound A:

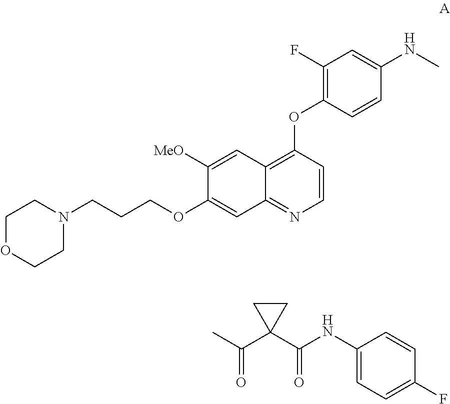

or a pharmaceutically acceptable salt thereof, wherein the human has a complete response or partial response.

### FIELD OF THE INVENTION

The present invention relates to a method of treating hepatocellular carcinoma (HCC) in a human.

### BACKGROUND OF THE INVENTION

Hepatocellular carcinoma (HCC) is the sixth and eleventh most common cancer worldwide in men and women, respectively (Hussain, et al. Ann Oncol. 2001; 12:161-72). Globally, over 600,000 new cases are diagnosed each year, and it is the third leading cause of cancer mortality. The geographic areas at highest risk, with age-adjusted incidence rates of greater than 20 per 100,000, are China and eastern Asia, middle Africa, and some countries of western Africa. Moderately high incidences (10 to 20 per 100,000) are found in Japan, southern Europe, Switzerland, and Bulgaria, whereas the lowest risk areas include northern Europe, Australia, New Zealand, and in the Caucasian population in North and Latin America (Lopez J B. Clin Biochem Rev. 2005; 26:65-9).

The majority of HCC cases occur in males, although the male-to-female ratio is far more striking in African and Asian patients (4:1 to 8:1) than in patients in low-incidence regions (2:1 to 3:1). Populations with an intermediate risk of HCC generally have a ratio of about 4:1. The difference in incidence by gender is thought to be contributed to by variations in hepatitis carrier states, differential exposure to environmental toxins, and the trophic effect of androgens (Okuda K. Epidemiology of primary liver cancer. In: Tobe T, editor. Primary liver cancer in Japan. Tokyo: Springer-Verlag; 1992:3).

Known risk factors for HCC include the hepatitis B carrier state, chronic hepatitis C infection, environmental toxins (e.g., aflatoxin), hereditary hemochromatosis, acute and chronic hepatic porphyria, and cirrhosis from any cause (most commonly alcohol). Hepatocellular carcinoma occurs most often in patients over 40 years of age [Lopez supra 2005], consistent with the association of HCC with long-standing liver disease. The prognosis of HCC is generally grave due to local progression and/or metastasis. In the Chinese and African populations, the mean survival time may be as short as 11 weeks from the onset of symptoms and 6 weeks from the time of diagnosis. Comparatively, the disease progresses somewhat more slowly in patients in low-risk regions, although even they have a mean survival of only about 6 months (Lopez supra, 2005]. The degree of hepatic dysfunction produced by the various causes of HCC is the most likely reason for the difference in biologic aggressiveness of the disease. Most patients with HCC also suffer from liver cirrhosis; thus, the malignant disease as well as the treatment may jeopardize the fragile balance of liver function.

The receptor for hepatocyte growth factor (HGF), known as mesenchymal epithelial transition factor (c-MET), is a receptor tyrosine kinase (RTK) widely expressed in epithelial and endothelial cells. Its cognate ligand, HGF, is expressed by cells of the mesenchymal lineage, facilitating rigorous regulation of c-MET kinase activity. The signaling of Hgf/c-MET genes is involved in hepatic development and biology in several ways (Michalopoulos G K, DeFrances M C. Liver regeneration. Science. 1997; 276:60-6). Hepatocyte growth factor is a potent mitogen for hepatocytes. Its proliferative effect on hepatocytes and HCC cells is mediated through c-MET. Increased HGF levels after partial hepatectomy promote liver regeneration by enhancing proliferation of mature hepatocytes and hepatic progenitor cells. In patients with HCC, high peripheral and portal HGF serum levels are associated with poor prognosis after hepatic resection (Chau, et al. Eur J Surg Oncol. 2008:34:333-8). In Hgf knockout mice, the hepatic plate is underdeveloped (Schmidt, Nature. 1995; 373:699-702). In adult rat livers, c-MET activation has been shown to alleviate chemically induced fibrosis [Ueki, Nat. Med. 1999; 5:226-30] and to protect hepatocytes from CD95-mediated apoptosis. c-MET is a central mediator of cell growth, survival, motility, and morphogenesis during early development. However, its natural role in adults appears to be primarily confined to repair/regeneration following injury of tissues such as liver (Birchmeier, et al. Nat Rev Mol Cell Biol. 2003; 4:915-25). Hepatocyte growth factor and c-MET may thus be effective targets for therapy in HCC.

The c-MET receptor has been implicated as a mediator in many important aspects of tumor pathobiology including tumor survival, growth, angiogenesis, invasion, and dissemination (Birchmeier, et al. Nat Rev Mol Cell Biol. 2003; 4:915-25; Ma, et al. Cancer Res. 2003a; 63:6272-81). The vascular endothelial growth factor (VEGF) receptor VEGFR2 (kinase insert domain receptor [KDR]) is also a central mediator of tumor angiogenesis. In addition to their individual roles in tumor pathobiology, preclinical data suggest that c-MET and VEGFR2/KDR play synergistic roles in promoting tumor angiogenesis and subsequent dissemination (Bottarro and Liotta, Nature. 2003; 423:593-5). Sorafenib (Nexavar, Bayer), a tyrosine kinase inhibitor that inhibits VEGFRs and BRAF (v-raf murine sarcoma viral oncogene homolog B1), has been shown to prolong stable disease (SD) (albeit with minimal tumor shrinkage) (Abou-Alfa, et al. J Clin Oncol. 2006; 24:4293-300). This activity has translated into survival advantage compared with placebo in the first-line treatment of HCC (Llovet, et al. J Clin Oncol 2007 ASCO Annual Meeting Proceedings. 2007; 25(20 Jun Suppl):LBA1). Preclinical data support a central role for c-MET in tumor pathobiology. The proto-oncogene c-MET regulates metastasis formation, tumor invasion, and angiogenesis [Ma, et al. Cancer Metastasis Rev. 2003b; 22:309-25]. Amplification, activating mutations, and overexpression of c-MET have been associated with poor prognosis and metastatic phenotype in a variety of human cancers such as papillary renal cell carcinoma (PRC) and gastric cancer [Ma, et al. Cancer Metastasis Rev. 2003b; 22:309-25).

Dysregulation of c-MET signaling results in enhanced tumorigenicity and metastatic potential in engineered cells and in transgenic mice (reviewed in Birchmeier, 2003 supra; Ma, 2003a supra). Conversely, inhibition of c-MET expression by use of ribozymes or antisense RNA inhibits growth of diverse human tumor xenografts in mice (Abounader, et al, FASEB J. 2002; 16:108-10; Kim, et al. Clin Cancer Res. 2003; 9:5161-70; Stabile, et al. Gene Ther. 2004; 11:325-35). Additionally, neutralizing antibodies to HGF inhibit the growth of human glioblastoma xenografts in mice, and administration of NK4, the novel antagonistic variant of HGF, inhibits orthotropic growth, invasion, and metastasis of human pancreatic carcinoma cells in mice (Cao, Proc Natl Acad Sci USA. 2001; 98:7443-8; Tomioka, Cancer Res. 2001; 61:7518-24).

Activation and/or overexpression of c-MET have been widely documented as frequent events in all major human tumor types (reviewed in Birchmeier, et al. 2003; Ma, et al. 2003a). In human HCC, overexpression and mutation of the c-MET gene are associated with intrahepatic metastases and vascular invasion (Corso, et al. Trends Mol. Med. 2005; 11:284-92). Expression of c-MET has been consistently correlated with more aggressive disease and poor prognosis (D'Errico, et al. Hepatology. 1996; 24:60-4; Daveau, et al. Mol. Carcinog. 2003; 36:130-41; Ueki, et al. Hepatology. 1997; 25:862-6.). Moreover, inhibition of c-MET by several therapeutic strategies including tyrosine kinase inhibitors [Wang, et al. J. Hepatol. 2004; 41:267-73), small interfering RNAs (Zhang, et al. Mol Cancer Ther. 2005; 4:1577-84), and gene therapy (Heideman, et al. Cancer Gene Ther. 2005; 12:954-62) have shown promise in the treatment of in vitro and in vivo preclinical models of HCC. These data validate c-MET as a therapeutic target in HCC. There are few specific c-MET inhibitors under clinical development, and none have so far been tested in the setting of HCC. Another RTK, the receptors for angiopoietin-1 and -2, has also been associated with HCC initiation and progression (Zhang, et al. World J. Gastroenterol. 2006; 12:4241-5), and multiple anti-RTK therapeutic strategies have shown promise in the treatment of HCC in preclinical models. c-MET is thus regarded as a promising molecular target for antimetastatic therapies (Chen, et al. Hepatology. 1997; 26:59-66).

Foretinib (also referred to as Compound A herein) is an oral multikinase inhibitor targeting c-Met, Tie-2, RON, Axl, and VEGFR—. HGF/Met signaling plays a pivotal role in tumor cell proliferation, migration and invasion and circulating levels of HGF correlate with poor prognosis in HCC. Compounds that simultaneously inhibit VEGF and c-MET RTKs may be more effective anticancer agents than agents targeting each of these receptors individually (Pennacchietti, et al. Cancer Cell. 2003; 3:347-61. 2003). In addition, foretinib has activity against other RTKs that have been implicated in tumor pathobiology, including the transmembrane tyrosine kinase KIT, platelet-derived growth factor receptors, FMS-like tyrosine kinase 3, and the receptor for angiopoietin-2, Tie-2.

It would be useful to provide novel methods of treatment for an individual suffering from hepatocellular carcinoma wherein the individual shows complete response, partial response and/or no disease progression.

### SUMMARY OF THE INVENTION

Methods are provided for treating a human having heptocellular carcinoma comprising administering to the human a therapeutically effective amount of Compound A:

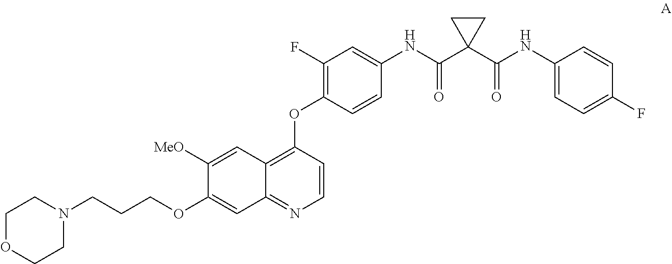 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt thereof, wherein the human has a complete response or partial response.

### DETAILED DESCRIPTION OF THE INVENTION

In one aspect, methods are provided for treating a human having heptocellular carcinoma comprising administering to said human a therapeutically effective amount of Compound A:

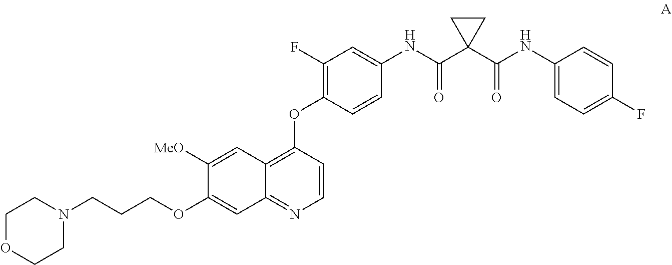 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt thereof, wherein said human has a complete response or partial response. In one aspect, the human has complete response. In another aspect the human has a partial response. Complete response and/or partial response can be measured by modified (mRECIST) or RECIST 1.0 criteria.

In one aspect, Compound A is administered as a free base. Compound A can be administered at a dose of at least 7.5 mg daily. Compound A can be administered, for instance, at a dose of about 7.5 mg, 15.0 mg, 30.0 mg and/or 45.0 mg daily. Compound A may be provided in tablet form. In some instances, tablets comprise hypromellose, sodium lauryl sulfate, lactose monohydrate, microcrystalline cellulose, croscarmellose sodium, and magnesium stearate. Some tablets may comprise hypromellose, titanium dioxide, polyethylene glycol. Tablets may comprise solysorbate 80 and iron oxide yellow.

In some aspects, the human has hepatocellular carcinoma that is unresectable or metastatic. In one aspect, the human has not previously received another multiple receptor tyrosine kinase inhibitor. In yet another embodiment Compound A is administered as monotherapy.

As used herein, the term “effective amounts” means amounts of the drugs or pharmaceutical agents that will elicit the desired biological or medical response of a tissue, system, animal, or human. Furthermore, the term “therapeutically effective amounts” means any amounts which, as compared to a corresponding subject who has not received such amounts, results in improved treatment, healing, prevention, or amelioration of a disease, disorder, or side effect, or a decrease in the rate of advancement of a disease or disorder. The term also includes within its scope amounts effective to enhance normal physiological function. It is to be understood that the compounds can be administered sequentially or substantially simultaneously.

The compounds of the present invention may exist in crystalline or non-crystalline form, or as a mixture thereof. The skilled artisan will appreciate that pharmaceutically acceptable solvates may be formed for crystalline compounds wherein solvent molecules are incorporated into the crystalline lattice during crystallization. Solvates may involve non-aqueous solvents such as ethanol, isopropanol, DMSO, acetic acid, ethanolamine, and ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The present invention includes all such solvates and forms.

The present invention includes compounds as well as their pharmaceutically acceptable salts. The word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds of the method of the present invention herein may be prepared. These pharmaceutically acceptable salts may be prepared in situ during the final isolation and purification of the compound, or by separately reacting the purified compound in its free acid or free base form with a suitable base or acid, respectively.

Compound A (also referred to herein as N¹-{3-fluoro-4-[(6-(methyloxy)-7-{[3-(4-morpholinyl)propyl]oxy}-4-quinolinyl)oxy]phenyl}-N¹-(4-fluorophenyl)-1,1-cyclopropanedicarboxamide), is disclosed and claimed, along with pharmaceutically acceptable salts and solvates thereof, methods of preparation, and as being useful as an inhibitor of cMET, particularly in treatment of cancer, in International Application No. PCT/US2004/031523, having an International filing date of Sep. 24, 2004; International Publication Number WO2005/030140 and an International Publication date of Apr. 7, 2005, the entire disclosure of which is hereby incorporated by reference. Examples 25 (p. 193), 36 (pp. 202-203), 42 (p. 209), 43 (p. 209), and 44 (pp. 209-210) describe how Compound A can be prepared. Compound A can be prepared as described in International Application No. PCT/US2009/064341 having an International filing date of Nov. 13, 2008; International Publication Number WO2010/056960 and an International Publication date of May 20, 2010, the entire disclosure of which is hereby incorporated by reference and in International Application No. PCT/US2009/058276 having an International filing date of Sep. 25, 2009; International Publication Number WO2010/036831 and an International Publication date of Apr. 1, 2010 the entire disclosure of which is hereby incorporated by reference.

The general preparation for Compound A is outlined in Scheme 1:

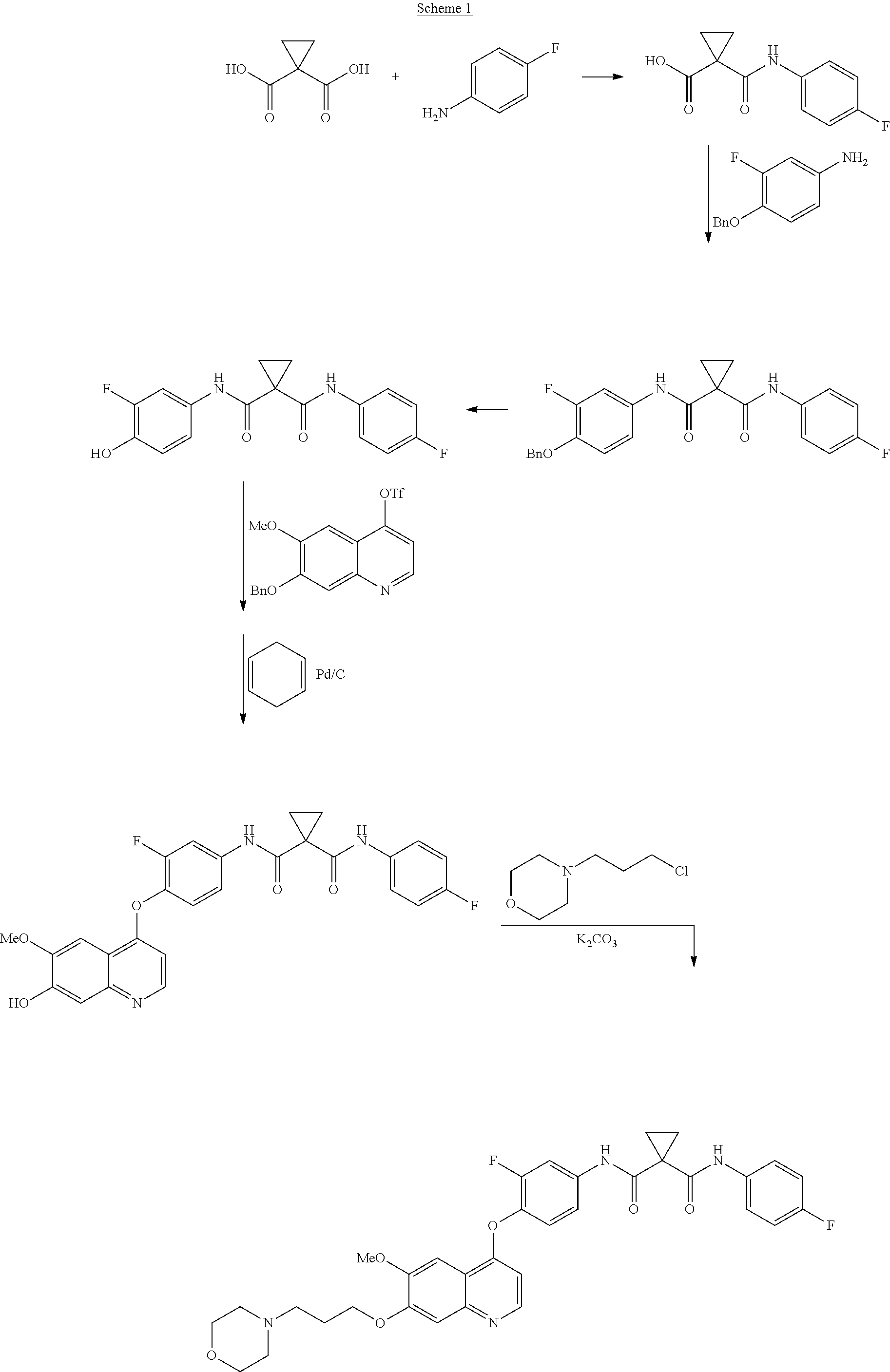

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; receptor tyrosine kinase inhibitors; serine-threonine kinase inhibitors; non-receptor tyrosine kinase inhibitors; angiogenesis inhibitors, immunotherapeutic agents; proapoptotic agents; and cell cycle signalling inhibitors.

The present invention also provides methods for treating cancer comprising administering Compound A or pharmaceutically acceptable salt thereof with or without another anti-neoplastic agent (Compound B).

By the term “specified period” and grammatical variations thereof, as used herein is meant the interval of time between the administration of one of Compound A² and Compound B² and the other of Compound A² and Compound B². Unless otherwise defined, the specified period can include simultaneous administration. Unless otherwise defined the specified period refers to administration of Compound A² and Compound B² during a single day.

By the term “duration of time” and grammatical variations thereof, as used herein is meant a compound of the invention is administered for an indicated number of consecutive days. Unless otherwise defined, the number of consecutive days does not have to commence with the start of treatment or terminate with the end of treatment, it is only required that the number of consecutive days occur at some point during the course of treatment.

Examples of a further active ingredient or ingredients (anti-neoplastic agent) for use in combination or co-administered with Compound A or pharmaceutically acceptable salt thereof are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, equation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthracyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-Iyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-Iyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (6-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Pazopanib which commercially available as VOTRIENT® is a tyrosine kinase inhibitor (TKI). Pazopanib is presented as the hydrochloride salt, with the chemical name 5-[[4(2,3-dimethyl-2H-indazol-6-yl)methylamino]-2-pyrimidinyl]amino]-2-methylbenzenesulfonamide monohydrochloride. Pazoponib is approved for treatment of patients with advanced renal cell carcinoma.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

mTOR inhibitors include but are not limited to rapamycin (FK506) and rapalogs, RAD001 or everolimus (Afinitor), CCl-779 or temsirolimus, AP23573, AZD8055, WYE-354, WYE-600, WYE-687 and Pp121.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl)ethenyl]benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino]phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)pethyl]amino}methyl)-2-furanyl]-4-quinazolinamine (represented by formula II, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

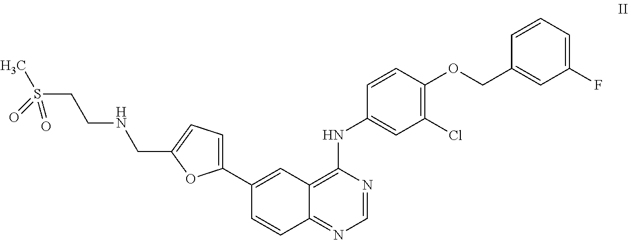 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (II) may be prepared according to the procedures disclosed in WO 99/35146, published Jul. 15, 1999; and WO 02/02552 published Jan. 10, 2002.

Erlotinib, N-(3-ethynylphenyl)-6,7-bis{[2-(methyloxy)ethyl]oxy}-4-quinazolinamine (commercially available under the tradename Tarceva) is represented by formula III, as illustrated:

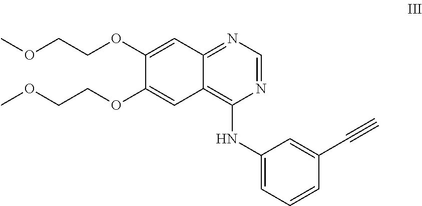 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine, N-(3-chloro-4-fluorophenyl)-7-methoxy-6-[3-4-morpholin)propoxy] is represented by formula IV, as illustrated:

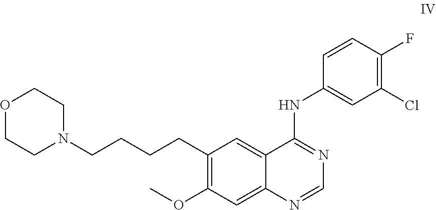 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As is understood in the art, the terms “complete remission,” “complete response” and “complete regression” mean the disappearance of all detectable signs and/or symptoms of cancer in response to treatment. As is also understood in the art detectable signs or symptoms of cancer can be defined based on the type and stage of cancer being treated. By way of example, “complete response” to treatment in a subject suffering from HCC could be defined as no visible liver tumors observed with X-ray or CT scan. In some instances, clinical response can be defined by RECIST 1.0 criteria (Therasse P, Arbuck S G, Eisenhauer E A, Wanders J, Kaplan R S, Rubinstein L, et al. New guidelines to evaluate the response to treatment in solid tumors. European Organization for Research and Treatment of Cancer, National Cancer Institute of the United States, National Cancer Institute of Canada. J Natl Cancer Inst. 2000; 92:205-16.) as described below:

### Recist 1.0 Criteria

### Definition of Measurable and Non-Measurable Disease

### Measurable Disease:

The presence of at least one measurable lesion.

### Measurable Lesion:

Lesions that can be accurately measured in at least one dimension, with the longest diameter (LD) being:
≧20 mm with conventional techniques (medical photograph [skin or oral lesion], palpation, plain X-ray, CT, or MRI),
OR
≧10 mm with spiral CT scan.

Non-measurable lesion: All other lesions including lesions too small to be considered measurable (longest diameter <20 mm with conventional techniques or <10 mm with spiral CT scan) including bone lesions, leptomeningeal disease, ascites, pleural or pericardial effusions, lymphangitis cutis/pulmonis, abdominal masses not confirmed and followed by imaging techniques, cystic lesions, or disease documented by indirect evidence only (e.g., by lab values).

### Methods of Measurement

### Conventional CT and MRI:

Minimum sized lesion should be twice the reconstruction interval. The minimum size of a baseline lesion may be 20 mm, provided the images are reconstructed contiguously at a minimum of 10 mm. MRI is preferred, and when used, lesions must be measured in the same anatomic plane by use of the same imaging sequences on subsequent examinations. Whenever possible, the same scanner should be used.

### Spiral CT:

Minimum size of a baseline lesion may be 10 mm, provided the images are reconstructed contiguously at 5 mm intervals. This specification applies to the tumors of the chest, abdomen, and pelvis.

### Chest X-Ray:

Lesions on chest X-ray are acceptable as measurable lesions when they are clearly defined and surrounded by aerated lung. However, MRI is preferable.

### Clinical Examination:

Clinically detected lesions will only be considered measurable by RECIST criteria when they are superficial (e.g., skin nodules and palpable lymph nodes). In the case of skin lesions, documentation by color photography—including a ruler and patient study number in the field of view to estimate the size of the lesion—is required.

### Baseline Documentation of Target and Non-Target Lesions

All measurable lesions up to a maximum of five lesions per organ and ten lesions in total, representative of all involved organs, should be identified as target lesions and recorded and measured at baseline.

Target lesions should be selected on the basis of their size (lesions with the LD) and their suitability for accurate repeated measurements (either clinically or by imaging techniques).

A sum of the LD for all target lesions will be calculated and reported as the baseline sum LD. The baseline sum LD will be used as a reference by which to characterize the objective tumor response.

All other lesions (or sites of disease) should be identified as non-target lesions and should also be recorded at baseline. Measurements of these lesions are not required, but the presence or absence of each should be noted throughout follow-up.

Documentation of indicator lesion(s) should include date of assessment, description of lesion site, dimensions, and type of diagnostic study used to follow lesion(s).

All measurements should be taken and recorded in metric notation, using a ruler or callipers.

### Response Criteria

Disease assessments are to be performed every 6 weeks after initiating treatment. However, subjects experiencing a partial or complete response must have a confirmatory disease assessment at least 28 days later. Assessment should be performed as close to 28 days later (as scheduling allows), but no earlier than 28 days.

Definitions for assessment of response for target lesion(s) are as follows:

### Evaluation of Target Lesions

Complete Response (CR)—disappearance of all target lesions.

Partial Response (PR)—at least a 30% decrease in the sum of the LD of target lesions, taking as a reference, the baseline sum LD.

Stable Disease (SD)—neither sufficient shrinkage to qualify for PR nor sufficient increase to qualify for progressive disease (PD), taking as a reference, the smallest sum LD since the treatment started. Lesions, taking as a reference, the smallest sum LD recorded since the treatment started or the appearance of one or more new lesions.

### Evaluation of Non-Target Lesions

Definitions of the criteria used to determine the objective tumor response for non-target lesions are as follows:

Complete Response—the disappearance of all non-target lesions.
Incomplete Response/Stable Disease—the persistence of one or more non-target lesion(s).
Progressive Disease—the appearance of one or more new lesions and/or unequivocal progression of existing non-target lesions.

### Evaluation of Overall Response for RECIST-Based Response

The overall response is the best response recorded from the start of the treatment until disease progression/recurrence is documented. In general, the subject's best response assignment will depend on the achievement of both measurement and confirmation criteria.

The following table presents the evaluation of best overall response for all possible combinations of tumor responses in target and non-target lesions with or without the appearance of new lesions.

| Target | | | |
|---|---|---|---|
| Lesion | Non-Target Lesion | New Lesion | Overall response |
| CR | CR | No | CR |
| CR | Incomplete response/(SD) | No | PR |
| PR | Non-PD | No | PR |
| SD | Non-PD | No | SD |
| PD | Any | Yes or No | PD |
| Any | PD | Yes of No | PD |
| Any | Any | Yes | PD |

Note:
Subjects with a global deterioration of health status requiring discontinuation of treatment without objective evidence of disease progression at that time should be classified as having “symptomatic deterioration”. Every effort should be made to document the objective progression even after discontinuation of treatment. In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Confirmation Criteria

To be assigned a status of PR or CR, a confirmatory disease assessment should be performed no less than 28 days after the criteria for response are first met.

To be assigned a status of SD, follow-up measurements must have met the SD criteria at least once after study entry at a minimum interval of 12 weeks.

### OTHER DEFINITIONS

By the term “combination” and grammatical variations thereof, as used herein is meant either simultaneous administration or any manner of separate sequential administration of a therapeutically effective amount of Compound A, or a pharmaceutically acceptable salt thereof, and Compound B or a pharmaceutically acceptable salt thereof. Furthermore, it does not matter if the compounds are administered in the same dosage form, e.g. one compound may be administered topically and the other compound may be administered orally. Suitably, both compounds are administered orally.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Tumors may be solid tumors such as HCC lesions. Tumors may be hematopoietic tumor, for example, tumors of blood cells or the like, meaning liquid tumors.

As used herein “overexpressed” and “overexpression” of a protein or polypeptide and grammatical variations thereof means that a given cell produces an increased number of a certain protein relative to a normal cell. By way of example, a c-Met protein may be overexpressed by a tumor cell relative to a non-tumor cell. Additionally, a mutant c-Met protein may be overexpressed compared to wild type c-Met protein in a cell. As is understood in the art, expression levels of a polypeptide in a cell can be normalized to a housekeeping gene such as actin. In some instances, a certain polypeptide may be underexpressed in a tumor cell compared with a non-tumor cell.

As used herein the term “amplification” and grammatical variations thereof refers to the presence of one or more extra gene copies in a chromosome complement. In certain embodiments a gene encoding a c-Met protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

The term “at least one mutation” in a polypeptide or a gene encoding a polypeptide and grammatical variations thereof means a polypeptide or gene encoding a polypeptide having one or more allelic variants, splice variants, derivative variants, substitution variants, deletion variants, truncation variants, and/or insertion variants, fusion polypeptides, orthologs, and/or interspecies homologs. By way of example, at least one mutation of a c-Met protein would include a c-Met protein in which part of all of the sequence of a polypeptide or gene encoding the c-Met protein is absent or not expressed in the cell for at least one c-Met protein produced in the cell. For example, a c-Met protein may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type. By way of another example a mutation in a c-Met protein would include a c-Met s protein having one or more amino acid differences in its amino acid sequence compared with wild type of the same c-Met protein. Mutation may be somatic or germline.

The following examples are intended for illustration only and are not intended to limit the scope of the invention in any way.

### Example 1

Tumor assessments were performed according to The Response Evaluation Criteria in Solid Tumours (RECIST). These criteria were proposed in 2000 (Therasse, et al. J Natl Cancer Inst. 2000; 92:205-16) and have enjoyed a wide adoption for the evaluation of solid tumors in oncology trials, providing a surrogate for clinical benefit particularly for cytotoxic chemotherapy agents.

However, the RECIST system is considered of limited value to evaluate response, progression and the presence of new lesions in HCC [Llovet, et al., J Natl Cancer Inst. 2008, 100:698-711) and a modification of RECIST for evaluation of HCC has been proposed by the European Association for the Study of the Liver and the American Association for the Study of Liver Disease (AASLD), (Bruix™, et al. J. Hepatol. 2001; 35:421-30 and Bruix, et al. Hepatology 2005; 42:1208-1236). These proposals have led to a new set of guidelines for the conduct of HCC clinical trials from a panel of HCC experts convened by the AASLD in December 2006. The guidelines introduce the concept of ‘typical’ intrahepatic HCC lesions (i.e. lesions displaying characteristic vascular patterns on dynamic contrast enhanced spiral CT or MRI imaging) and differentiation between viable and necrotic tumor tissue when assessing response. For ‘atypcial’ and extrahepatic lesions the original RECIST guidelines are followed. Retrospective analysis of the brivanib phase-II, advanced HCC studies supports the value of modified RECIST (mRECIST) for prediction of clinical benefit with targeted agents in advanced HCC (Finn, et al., J Clin Oncol 2010; 28:15s, (suppl; abstr 4096)). The guidelines have also been implemented in ongoing phase III trials of targeted agents in advanced HCC and the validity of this surrogate endpoint will be prospectively evaluated.

To enable a more accurate description of the potential clinical benefit of foretinib in patients with HCC, the method of tumor evaluation was changed to incorporate the mRECIST criteria for HCC as proposed by Llovet et al (Llovet, et al. 2008 supra) and further described by Lencioni & Llovet (Lencioni & Llovet Modified RECIST (mRECIST) Assessment for Hepatocellular Carcinoma. Seminars in Liver Disease. 2010; 30:52-60) in addition to the standard RECIST assessment.

The primary endpoint for assessing antitumor activity will be response rate (CR+PR) according to mRECIST (Lencioni, 2010), assessed by independent, central, radiological review, in subjects with advanced (unresectable and/or metastatic) HCC treated with foretinib at the maximum tolerated dose.

Additional antitumor activity endpoints will include the following:
Time to progression of subjects with advanced (unresectable and/or metastatic) HCC treated with foretinib at the maximum tolerated dose. Disease progression is defined as objective disease progression according to mRECIST, assessed by independent, central, radiological review, and/or clinical (e.g., symptomatic) progression.
Duration of response according to mRECIST in subjects with advanced (unresectable and/or metastatic) HCC treated with foretinib at the maximum tolerated dos.
Percentage of subjects with advanced (unresectable and/or metastatic) HCC treated with foretinib at the maximum tolerated dose who have a baseline serum alpha fetoprotein measurement of at least 200 ng/mL and achieve a 50% decrease from baseline while on study treatment.

Each treatment cycle will consist of 21 consecutive days. Subjects enrolled in this study will receive oral foretinib administered once daily. Blood samples for PK analysis will be obtained before and after dosing on Days 1 and 15 of Treatment Period 1 in both the Dose-Escalation and Expanded Cohort Phases. For subjects in the Expanded Cohort Phase, no dose will be administered on Days 2 and 3 of Treatment Period 1 to allow the pharmacokinetics of foretinib to be assessed over a 72 hour interval after the first dose. Subjects will continue to receive study drug until disease progression or withdrawal from study because of unacceptable toxicity or other reasons (e.g., withdrawal of consent, noncompliance). Disease progression is defined as objective disease progression according to mRECIST and/or clinical progression (e.g. emergence of significant new disease-related symptoms and/or significant deterioration of pre-existing disease-related symptoms requiring treatment; or deterioration of ECOG performance status by 2 units—see Appendix 3). For the purpose of the final analysis, the duration of the study is planned to be 2 years after enrollment is completed.

Tumor response for subjects with measurable lesions should be assessed routinely as specified in the table below. Serial CT-scans (preferred) or MRI will be evaluated for response (CR or PR) and SD according to mRECIST (Lecioni, 2010) and RECIST 1.0 (Therasse, et al. New guidelines to evaluate the response to treatment in solid tumors. European Organization for Research and Treatment of Cancer, National Cancer Institute of the United States, National Cancer Institute of Canada. J Natl Cancer Inst. 2000; 92:205-16.).

The same assessment method (MRI or CT scan) should be used to assess a lesion before, during and after treatment. The secondary study endpoints (response rate, time to progression, and duration of response) will be assessed by central, independent, radiological review using mRECIST. As an exploratory objective, investigators will also assess the secondary study endpoints by RECIST 1.0. Investigators may also assess subjects using mRECIST, but these data will not be collected on the eCRF. In addition, serial serum alpha fetoprotein measurements will be assessed as a secondary antitumor activity endpoint.

### Tumor Scanning Criteria

| Pre-treatment screening period | |
|---|---|
| Study treatment period | Study Treatment Period |
| Within 28 days of the first dose of | CT-scans (preferred) or MRI of |
| Foeretinib: | sites of disease every 6 weeks |
| CT-scans (preferred) or MRI of | Any additional imaging as clinically |
| chest, abdomen and pelvis | indicated |
| Any additional imaging as | Confirmation of response: not less |
| clinically indicated | than 28 days after first |
| | documentation of response |

To be assigned a status of confirmed PR or CR, changes in tumor measurements must be confirmed by repeat studies performed at least 28 days after the criteria for response are first met. In the case of SD, follow-up measurements must have met the SD criteria at least once after study entry at a minimum interval of 12 weeks from initiation of therapy.
The Same Diagnostic Method Must be Used Throughout the Study to Evaluate a Lesion. Conventional CT and MRI:

Minimum sized lesion should be twice the reconstruction interval. The minimum size of a baseline lesion may be 20 mm, provided the images are reconstructed contiguously at a minimum of 10 mm. CT is preferred, and when used, lesions must be measured in the same anatomic plane by use of the same imaging sequences on subsequent examinations. Whenever possible, the same scanner should be used.

Spiral CT:

Minimum size of a baseline lesion may be 10 mm, provided the images are reconstructed contiguously at 5 mm intervals. This specification applies to the tumors of the chest, abdomen, and pelvis.

### Chest X-Ray:

Lesions on chest X-ray are acceptable as measurable lesions when they are clearly defined and surrounded by aerated lung. However, MRI is preferable.

### Clinical Examination:

Clinically detected lesions will only be considered measurable by RECIST criteria when they are superficial (e.g., skin nodules and palpable lymph nodes). In the case of skin lesions, documentation by color photography—including a ruler and patient study number in the field of view to estimate the size of the lesion—is required.

### TARGET LESION DEFINITION

### ‘Typical’ Target Lesions

Intrahepatic lesions that meet the following criteria are considered ‘typical’ target lesions:
Lesions that show typical features of HCC in contrast-enhanced spiral CT or MRl studies (i.e. hypervascularity in the arterial phase with wash-out in the portal or late venous phase)
Lesions that are measurable (as defined above)
Lesions that are suitable for repeated measurements

### Other Target Lesions (‘Atypical’ and Extrahepatic Lesions)

‘Atypical’ and extrahepatic lesions that are measurable and suitable for repeated measurements are considered target lesions

### Target Lesion Selection

The selection of target lesions will be guided initially by the presence of ‘typical’ intrahepatic lesions:

If ‘Typical’ intrahepatic lesions are present:

Up to 5 of these ‘typical’ intrahepatic lesions should be selected as target lesions at baseline. Measurement of viable tumor diameter will be applied to these lesions. All intrahepatic lesions beyond these 5 should be considered as non-target lesions.

In addition, up to 5 measurable, extrahepatic lesions per organ should be selected as target lesions at baseline. Measurement of longest tumor diameter will be applied to these lesions. All extrahepatic lesions beyond the up-to-10 selected target lesions should be considered as non-target lesions.

If No ‘Typical’ Intrahepatic Lesions are Present:

In case measurable intrahepatic HCC lesions are present at baseline but do not meet the criteria for ‘typical’ lesions, due to atypical vascular patterns, these measurable intrahepatic lesions as well as measurable extrahepatic lesions should be assessed at baseline. Up to a maximum of 5 lesions per organ and 10 lesions in total, representative of all involved organs, should be identified as target lesions and recorded and measured. A measurement of longest diameter will be applied to these lesions.

### Non-Target Lesions

Measurable lesions other than the target lesions and all sites of non-measurable (evaluable) disease will be identified as non-target lesions. Measurements of these lesions are not required, but the presence or absence of each should be noted throughout follow-up. Non-target lesions may include:

### Intrahepatic Lesions:

Not well-delineated HCC lesions including infiltrative-type and diffuse HCC
HCC lesions with atypical contrast-agent enhancement patterns
HCC lesions showing local recurrence after previous loco-regional treatment without meeting the criteria for ‘viable’ lesions (i.e. lack of clear-cut hypervascular recurrence and/or well-delineation from the surrounding livertissue)
Portal vein tumor invasion and/or thrombosis
Porta hepatis lymph node(s) considered as malignant (i.e. >20 mm in the short axis)
Intrahepatic viable lesions in excess of the 5 lesions in the liver selected as target lesions

### Extrahepatic Lesions:

Extrahepatic lesions in excess of the 5 lesions per organ selected as target lesions
Non-measurable but evaluable disease (i.e. cutaneous or bone lesions, etc.)

### Calculation of Sum of Target Lesions

Documentation of indicator lesion(s) should include date of assessment, description oflesion site, dimensions, and type of diagnostic study used to follow lesion(s).
All measurements should be taken and recorded in metric notation, using a ruler or callipers.

### If ‘Typical’ Intrahepatic Lesions are Present:

The sum of the viable diameters of all ‘typical’ intrahepatic target lesions (up to 5 lesions total) and of the longest overall diameters of extrahepatic target lesions (5 per organ) up to a maximum of 10 target lesions in total will be calculated and reported as the baseline sum. This baseline sum will be used as the reference for determining tumor response. Note the longest viable diameter may not be on the same section of liver as the longest overall diameter.

### If No ‘Typical’ Intrahepatic Lesions are Present:

The sum of the longest overall diameters for all target lesions up to a maximum of 10 target lesions in total will be calculated and reported as the baseline sum of the longest diameters, which will be used as reference to characterize the objective tumor response according to RECIST 1.0.

In case of an initial tumor shrinkage, the smallest sum of (1) viable diameters (for ‘typical’ intrahepatic lesions) or of (2) longest diameters (for ‘atypical’ intra- and extrahepatic lesions) recorded following baseline will be used as reference to determine disease progression.

### Response Criteria

Disease assessments are to be performed every 6 weeks after initiating treatment. However, subjects experiencing a partial or complete response must have a confirmatory disease assessment at least 28 days later. Assessment should be performed as close to 28 days later (as scheduling allows), but no earlier than 28 days. Definitions for assessment of response for target lesion(s) are as follows:

Complete Response (CR)—both of the following criteria must be met:

For ‘typical’ intrahepatic target- and non-target lesions, complete disappearance of any intratumoral contrast-agent enhancement in the arterial phase of spiral CT or dynamic MRI

### AND

For ‘atypical’ intra- and extrahepatic target- and non-target lesions, complete disappearance of all evidence of target- and non-target lesions in spiral CT or MRI

### Partial Response (PR)

A decrease of >30% in the sum of the longest diameters of all target lesions (defined below), with the baseline sum of the longest diameters of all target lesions as reference. The “sum of the longest diameters of all target lesions” is defined as the sum of the following:

The sum of the longest viable diameters of ‘typical’ intrahepatic target lesions

### AND

The sum of the longest overall diameters of ‘atypical’ intra- and extrahepatic target lesions.

Stable Disease (SD)—neither sufficient shrinkage to qualify for PR nor sufficient increase to qualify for progressive disease (PD).

Progressive Disease (PD)—one or more of the following criteria must be met:

For ‘typical’ intrahepatic target lesions, increase of >20% in the sum of the longest viable diameters of target lesions, taking as reference the smallest sum of viable diameters of target lesions recorded since the treatment started

### OR

For ‘atypical’ intra- and extrahepatic lesions increase of >20% in the sum of the longest diameters of target lesions, taking as reference the smallest sum of the diameters of target lesions recorded since the treatment started

### OR

New hepatic lesion with the longest diameter of at least 10 mm with the vascular pattern characteristic for HCC, i.e. hypervascularization in the arterial phase with wash-out in the portal venous (or late venous) phase of contrast-enhanced spiral CT or MRI imaging

### OR

New hepatic lesion larger than 10 mm without the vascular pattern characteristic for HCC, but evidence of growth of at least 10 mm in subsequent scans. Note: individual radiological events will be adjudicated retrospectively as PD at the time when it was first detected by imaging techniques, if the criteria are fulfilled 20 mm) on subsequent radiological testing

### OR

Unequivocal progression of existing non-measurable lesions. However, any new or worsening of pre-existing effusion (ascites, pleural effusion, etc.) will not be considered progression unless there is cyto-pathological confirmation of malignancy

### OR

Appearance of one or more new extrahepatic lesions of any size

### OR

Unequivocal progression of existing intra- or extrahepatic non-target lesion(s)

In the absence of clinical progression or concurrent progression in target lesions, progression of non-target lesions that is equivocal should be confirmed by a repeat evaluation at 3-6 weeks. If the progression is confirmed, the date of the first (equivocal) assessment will be taken as the date of progression

Evaluation of Overall Response for mRECIST-Based Response

The overall response is the best response recorded from the start of the treatment until disease progression/recurrence is documented. In general, the subject's best response assignment will depend on the achievement of both measurement and confirmation criteria.

The following table presents the evaluation of best overall response for all possible combinations of tumor responses in target and non-target lesions with or without the appearance of new lesions.

| Target | | | |
|---|---|---|---|
| Lesion | Non-Target Lesion | New Lesion | Overall response |
| CR | CR | No | CR |
| CR | Incomplete response/(SD) | No | PR |
| PR | Non-PD | No | PR |
| SD | Non-PD | No | SD |
| PD | Any | Yes or No | PD |
| Any | PD | Yes of No | PD |
| Any | Any | Yes | PD |

Note:
Subjects with a global deterioration of health status requiring discontinuation of treatment without objective evidence of disease progression at that time should be classified as having “symptomatic deterioration”. Every effort should be made to document the objective progression even after discontinuation of treatment.

In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Confirmation Criteria

To be assigned a status of PR or CR, a confirmatory disease assessment should be performed no less than 28 days after the criteria for response are first met.

To be assigned a status of SD, follow-up measurements must have met the SD criteria at least once after study entry at a minimum interval of 12 weeks.

Assessment of Target Lesion Response: Conventional RECIST and mRECIST
Assessment for HCC Following the AASLD-JNCI Guideline

| RECIST | mRECIST for HCC |
|---|---|
| CR = Disappearance of all target lesions | CR = Disappearance of any intratumoral arterial |
| | enhancement in all target lesions |
| PR = At least a 30% decrease in the sum of | PR = At least a 30% decrease in the sum of |
| diameters of target lesions, taking as reference | diameters of viable(enhancement in the arterial |
| the baseline sum of the diameters of target | phase) target lesions, taking as reference the |
| lesions | baseline sum of the diameters of target lesions |
| SD = Any cases that do not qualify for either | SD = Any cases that do not qualify for either |
| partial response or progressive disease | partial response or progressive disease |
| PD = An increase of at least 20% in the sum of | PD = An increase of at least 20% in the sum of |
| the diameters of target lesions, taking as | the diameters of viable (enhancing) target |
| reference the smallest sum of the diameters of | lesions, taking as reference the smallest sum of |
| target lesions recorded since treatment started | the diameters of viable (enhancing) target |
| | lesions recorded since treatment started |

Lencioni & Llovet Modified RECIST (mRECIST) Assessment for Hepatocellular Carcinoma. Seminars in Liver Disease. 2010; 30: 52-60

### Results

Patients with measurable unresectable/metastatic HCC, no prior sorafenib or other multi-kinase inhibitors, ECOG PS 0-1, adequate organ function and Child-Pugh grade A are eligible for enrolment. Phase I is a standard 3+3 design using increasing doses of oral foretinib to evaluate safety and determine the maximum tolerated dose (MTD). Secondary objectives include antitumour activity in approximately 33 patients dosed at MTD and pharmacokinetics.

As of Oct. 23, 2011, 45 patients have been enrolled: median age 58 years (range 31-82 years), M/F=35/10, 100% Asian race, 82% with cirrhosis. Thirty-nine patients were dosed at 30 mg once daily (OD) and 6 patients were dosed at 45 mg OD. Adverse events (AE) were reported in 42 patients (93%). The most common AEs were hypertension (38%), peripheral oedema (18%), ascites (16%), decreased appetite (16%), hypoalbuminemia (16%), pyrexia (16%), alanine aminotransferase increased (13%), constipation (13%), diarrhea (11%), insomnia (11%), and thrombocytopenia (11%). Two dose-limiting toxicities (renal failure, proteinuria) were observed in 2/6 patients at 45 mg OD but no dose limiting toxicities were observed in 7 patients at 30 mg OD in Phase I. Thirty-milligrams OD was declared as the maximum tolerated dose. In 38 evaluable patients, dosed at the maximum tolerated dose the median time-to-progression (95% confidence intervals) using modified-RECIST was 4.5 months (2.8, not achieved). These results are similar to a similar population of first-line advanced HCC patients treated with sorafenib, where sorafenib showed median time to progression of 2.8 months (2.6, 3.6) [Cheng, A-L et al Lancet Oncol 2009; 10: 25-34]. Fifteen of the 38 evaluable patients are still receiving foretinib study drug. Exposure of 30 mg and 45 mg OD foretinib was overlapping and similar to higher doses in other tumor types.

### CONCLUSION

The maximum tolerated dose was determined to be foretinib 30 mg OD. The early promising signal of activity observed in this study needs to be confirmed later in additional studies.

1. A method of treating a human having heptocellular carcinoma comprising administering to said human a therapeutically effective amount of Compound A: 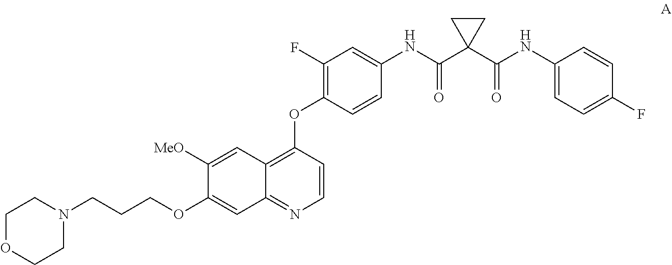 [SMILES: COc1cc2c(Oc3ccc(NC(=O)C4(C(=O)Nc5ccc(F)cc5)CC4)cc3F)ccnc2cc1OCCCN1CCOCC1]
or a pharmaceutically acceptable salt thereof, wherein said human has a complete response or partial response.

2. The method of claim 1, wherein said human has complete response.

3. The method of claim 1, wherein said human has partial response.

4. The method of claim 1, wherein said complete or partial response is measured by a modified RECIST criteria.

5. The method of claim 1, wherein said complete or partial response is measured by RECIST 1.0 criteria.

6. The method of claim 1, wherein Compound A is administered as a free base.

7. The method of claim 1, wherein Compound A is administered at a dose of at least 7.5 mg daily.

8. The method of claim 1, wherein Compound A is administered at a dose of at least about 30 mg daily.

9. The method of claim 1, wherein said hepatocellular carcinoma is unresectable or metastatic.

10. The method of claim 1, wherein said human has not previously received another multiple receptor tyrosine kinase inhibitor.

11. The method of claim 1, wherein said Compound A is administered as monotherapy.

12. A method of treating a human in need thereof comprising administering to said human 30 mg and/or 45 mg of Compound A, wherein said human has hepatocellular cancer.

13. (canceled)

14. (canceled)

15. A method inducing a complete response in a human comprising administering to said human a therapeutically effective amount of Compound A: 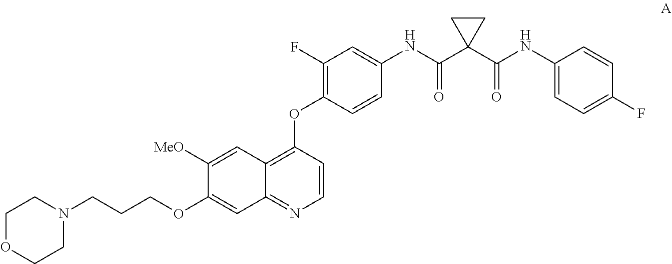 [SMILES: COc1cc2c(Oc3ccc(NC(=O)C4(C(=O)Nc5ccc(F)cc5)CC4)cc3F)ccnc2cc1OCCCN1CCOCC1]
or a pharmaceutically acceptable salt thereof, wherein said human has heptocellular carcinoma.
